(19) United States
(12) Patent Application Publication
(10) Pub. No.: US 2017/0304150 A1
(43) Pub. Date: Oct. 26, 2017

(54) STABLE PACKAGING SYSTEM FOR MOISTURE AND OXYGEN SENSITIVE PHARMACEUTICAL DOSAGE FORMS

(71) Applicant: Ascent Pharmaceuticals, Inc., Hauppauge, NY (US)

(72) Inventors: Sridhar Gumudavelli, Hauppauge, NY (US); Sudhakar Vidiyala, Hauppauge, NY (US); Madhava Reddy, Hauppauge, NY (US)

(21) Appl. No.: 15/490,216

(22) Filed: Apr. 18, 2017

Related U.S. Application Data
(60) Provisional application No. 62/324,472, filed on Apr. 19, 2016.

(51) Int. Cl. A61J 1/03; B65D 81/26; A61K 31/352; A61K 31/22; B01J 20/10; A61K 47/02; B65D 75/36

(52) U.S. Cl. CPC A61J 1/035; B65D 81/268; B65D 81/264; B65D 75/368; A61K 31/22; B01J 20/103; A61K 47/02; A61K 31/352; B01J 2220/66

(57) ABSTRACT

The invention describes a blister packaging system, and method of making the system, capable of establishing and maintaining arid and anaerobic conditions for packaging pharmaceutical products that are sensitive to oxygen and/or moisture. The blister packaging system encompasses, for example, a scaffold of several interconnected cavities intended for product and one or more reservoirs comprising an oxygen scavenger and/or a desiccant. Furthermore, the concentration of the components is maintained at a level that prevents establishment of equilibrium conditions.

This application claims the benefit of U.S. Provisional Application Ser. No. 62/324,472, filed Apr. 19, 2016, the entirety of which is incorporated herein by reference.

### BACKGROUND OF THE INVENTION

The present invention describes a blister packaging system, and method of making the system, capable of establishing and maintaining arid and anaerobic conditions for packaging pharmaceutical products that are sensitive to oxygen and/or moisture. The blister packaging system encompasses, for example, a scaffold of several interconnected cavities intended for product and one or more reservoirs comprising an oxygen scavenger and/or a desiccant. Furthermore, the concentration of the components is maintained at a level that prevents establishment of equilibrium conditions.

All references cited herein are incorporated herein by reference in their entireties.

### BRIEF SUMMARY OF THE INVENTION

The invention provides a packaging system for a pharmaceutical product that is sensitive to oxygen and/or moisture comprising: a blister pack comprising: (i) a blister sheet comprising of an array of interconnected cavities connected through channels that allows the exchange of air between them; (ii) wherein one or more of the cavities serve as a reservoirs holding an oxygen scavenger and/or a desiccant at concentrations high enough to provide higher equilibration time for oxygen and/or moisture; (iii) further wherein one or more of the cavities comprise at least one pharmaceutical product that is sensitive to oxygen and/or moisture; and (iii) a frangible lidding sealed to the sheet protecting the product in the cavity. The invention provides a package wherein the pharmaceutical product contains an active that comprises at least one amine as a part of chemical groups. The invention provides a package, where in the pharmaceutical products is an active that is selected from the group consisting of cannabinoids, drobinol, dronabinol, δ-tetrahydrocannabinol, cannabidiol, and combinations thereof.

The invention provides a package, wherein the pharmaceutical product is selected from the group consisting of statins, lovastatin, simvastatin, fluvastatin, atorvastatin, pitavastatin, and combinations thereof. The invention provides a package, wherein the pharmaceutical product is selected from the group consisting of morphine, hydromorphone, promethazine, dopamine, epinephrine, norepinephrine, esterified estrogen, ephedrine, pseudoephedrine, acetaminophen, ibuprofen, danofloxacin, erythromycin, penicillin, cyclosporine, methyldopate, cetirizine, diltiazem, verapamil, mexiletine, chlorothiazide, carbamazepine, selegiline, oxybutynin, vitamin A, vitamin B, vitamin C, L-cysteine, L-tryptophan, and combinations thereof.

The invention provides a package wherein the blister sheet that has light and moisture protectant properties. The invention provides a package wherein the blister sheet is made of materials selected from the group consisting of polyvinylchloride, polyvinylidene chloride, polycarbonate, polyester, copolyester, acrylonitrile, low density polyethylene, polypropylene, and combinations thereof. The invention provides a package wherein the oxygen scavenger is selected from the group consisting of organic, inorganic, metallic, non-metallic, and enzymatic in nature. The invention provides a package where in the desiccants is selected from the group consisting of organic desiccants, inorganic desiccants, uncoated polymers, coated adsorbent polymers, and combinations thereof. The invention provides a package wherein the frangible lidding has moisture barrier properties.

The invention provides a blister package for an oxygen and/or moisture sensitive pharmaceutical product comprising: (i) a shaped film comprising: (A) a plurality of cavities configured to each hold a single unit dose of the pharmaceutical product, and (B) at least one airflow channel, and (C) at least one reservoir; wherein the at least one airflow channel connects each of the plurality of cavities to the at least one reservoir; (ii) a frangible lidding sealed to the shaped film so that the unit dose is substantially confined between said frangible lidding and said at least one cavity; (iii) an absorbent confined between the reservoir and the frangible lidding; (iii) optionally a backing film; and (iv) single unit doses of an oxygen and/or moisture sensitive pharmaceutical product confined between the cavities and the frangible lidding. The invention provides a package wherein the absorbent comprises a desiccant and an oxygen absorber, wherein the desiccant is selected from the group consisting of silica gel, clay desiccants, calcium sulfate, calcium chloride, calcium oxide, zeolite, activated alumina, activated charcoal, and combinations thereof, and the oxygen absorber is selected from the group consisting of iron oxidase, glucose oxidase, sodium sulfate, potassium chloride, ammonium chloride, ammonium sulfate, calcium chloride, sodium phosphate, calcium phosphate, magnesium chloride, and combinations thereof.

The invention provides a package wherein the absorbent comprises a moisture sensitive color indicator to indicate when the desiccant is no longer operable. The invention provides a package wherein the cavities are interconnected by airflow channels. The invention provides a package wherein the pharmaceutical product is selected from the group consisting of morphine, hydromorphone, promethazine, dopamine, epinephrine, norepinephrine, esterified estrogen, ephedrine, pseudoephedrine, acetaminophen, ibuprofen, danofloxacin, erythromycin, penicillin, cyclosporine, methyldopate, cetirizine, diltiazem, verapamil, mexiletine, chlorothiazide, carbamazepine, selegiline, oxybutynin, vitamin A, vitamin B, vitamin C, L-cysteine, L-tryptophan, and combinations thereof. The invention provides a package wherein the shaped film comprises polyvinyl chloride, polyvinylidene chloride, polycarbonate, polyester, copolyester, acrylonitrile, low density polyethylene, polypropylene, or a combination thereof. The invention provides a package wherein an active pharmaceutical ingredient in the pharmaceutical product is an active that is selected from the group consisting of cannabinoids, drobinol, dronabinol, δ-tetrahydrocannabinol, cannabidiol, statins, lovastatin, simvastatin, fluvastatin, atorvastatin, pitavastatin, and combinations thereof.

### BRIEF DESCRIPTION OF SEVERAL VIEWS OF THE DRAWINGS

The invention will be described in conjunction with the following drawings in which like reference numerals designate like elements and wherein:

FIG. 1 shows an orthogonal view of an example of the blister pack component of the packaging system.

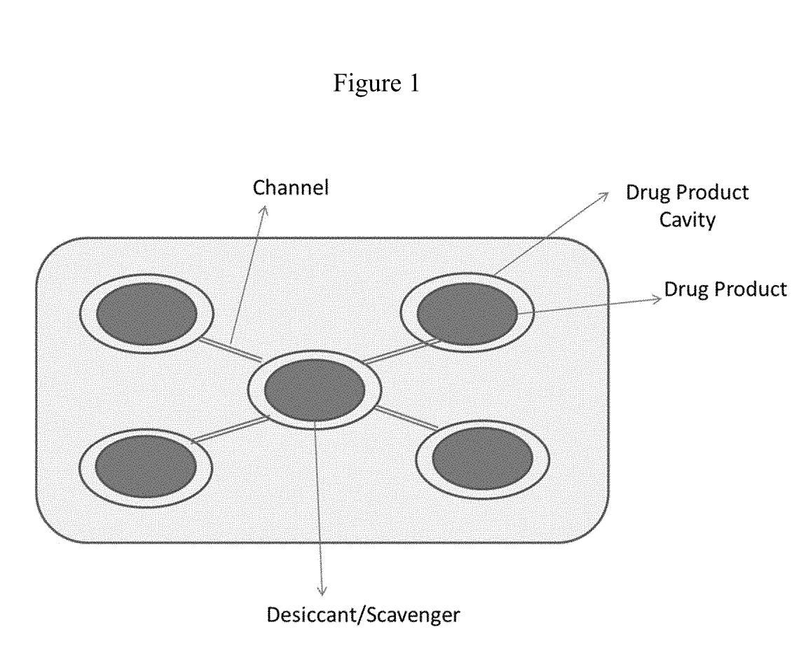

### DETAILED DESCRIPTION OF THE INVENTION

The present invention describes a blister packaging system capable of establishing and maintaining arid and anaerobic conditions for packaging pharmaceutical products that are sensitive to oxygen and/or moisture. The blister packaging system encompasses, for example, a scaffold of several interconnected cavities intended for product and one or more reservoirs comprising an absorbent which comprises an oxygen scavenger and/or a desiccant. The concentration of the components is maintained at a level that prevents establishment of equilibrium conditions.

The term “oxygen-sensitive pharmaceutical product” refers to any pharmaceutical product containing a substance that is prone to react with oxygen under normal ambient conditions (about 5° C. to about 40° C.). The reaction may involve the addition of oxygen to the substance, removal of hydrogen from the substance, or the loss or removal of one or more electrons from a molecular entity in the substance, with or without concomitant loss or removal of protons. It can also involve indirect processes where, for example, an oxidizing agent (e.g., peroxide, superoxide) is generated which oxidizes a substance in the pharmaceutical product.

The term “moisture-sensitive pharmaceutical product” refers to any pharmaceutical product containing a substance that is prone to degradation, crystal form conversion, physical instability and/or structural alteration under normal ambient conditions when water, water vapor and/or humidity are present. Thus, any pharmaceutical product containing a substance having a propensity for uptake of moisture, and the uptake unacceptably affects the physical properties or stability (dissolution, disintegration, hardness, friability) of the finished form of the product, is an example of a moisture-sensitive pharmaceutical product. The term also refers to any pharmaceutical product containing a substance affected by hydrolysis, whereby for example, a bond in the substance is cleaved by addition of hydrogen and hydroxide ions (ions resulting from the split of a water molecule).

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may easily be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

### Blister Packs

The term Blister packs herein is taken to mean packaging comprising at least two sheets, films, or foils which are firmly bonded to one another and which contain cavities for the accommodation of the pharmaceutical product to be packed. Blister packs usually consist of a thermoformed plastic sheet or film (cavity sheet, blister sheet, or shaped film) for the accommodation of the solids, which, after filling, is firmly bonded, e.g., heat-sealed, to a second sheet, film or foil (cover sheet, frangible layer, film or foil), which usually consists of an aluminium foil and/or plastic sheet or film. The packaged pharmaceutical product s can be pushed through the cover sheet, frangible layer, film or foil by pressure on the cavity sheet, film or foil and removed individually from the blister pack. Blister packs are therefore also known as push-through packs. If “pushing-through” the cover sheet, film or foil is not possible owing to the shape, size and/or strength of the solids contained, the blister packs can also be opened by slitting open the cover sheet, film or foil using a sharp object, for example using the finger nail. However, the term “blister pack” is not restricted thereto, but also encompasses special embodiments, such as, for example, child-proof modifications, such as, for example, those in which it is necessary to carry out two different opening operations whose sequences are intended to be beyond the intellectual capacity of children (such as so-called “peel-push systems”), or embodiments in which the cover sheet, film or foil is not punctured, but instead peeled off before removal of the solids contained.

Blister packs are the preferred primary packaging means for solid pharmaceutical administration forms. Advantages are that the administration forms can be removed individually and thus without contamination of the other administration forms, which are furthermore contained in sealed cavities, the administration forms are separated from one another (meaning that mutual interaction, such as, for example, abrasion or sticking, are basically prevented).

A further important function of blister packs is protection of the pharmaceutical administration forms contained therein against harmful environmental influences, such as light, gases, in particular oxygen, and against moisture. Since blister packs are usually accommodated in folding cartons, which are not effective barriers against moisture and gases, the crucial protective action in the case of solid pharmaceutical administration forms packaged in blister packs (primary packaging means) and folding cartons (secondary packaging means) arises through the blister packs.

Various plastic sheets or films, such as, for example, polyvinyl chloride (PVC), polyvinylidene chloride (PVDC), high-density polyethylene (HDPE), polypropylene (PP), polyethylene terephthalate (PET), polycarbonate, each of which have different material properties may be used in the blister packs of the invention. Although the selection of a material of relatively low permeability for moisture and/or oxygen or the use of composite sheets or films made from these materials, such as, for example, PVC/PVDC, PVC/HDPE, optionally also together with further polymers as barrier layer, such as, for example, cycloolefin copolymer (COC), or special polyhalogenated polymers, such as polychlorotrifluoroethylene (PCTFE) Aclar®, (PVC/PCTFE, PP/COC (for example Polybar®) PVC/COC/PVDC composite sheets or films), enables the ingress of moisture to be reduced to a certain degree, it does not prevent it completely.

Suitable for use for the manufacture of blister packs which are suitable for the pack according to the invention are all plastic sheets or films which can be converted into blister packs in corresponding plants, in particular thermoforming plants. Examples of plastic sheets and films which are suitable for the manufacture of blister packs are polyvinyl chloride (PVC), polyvinylidene chloride (PVDC), high-density polyethylene (HDPE), polypropylene (PP), polyethylene terephthalate (PET), polycarbonate, cycloolefin copolymer (COC), special polyhalogenated polymers, such as polychlorotrifluoroethylene (PCTFE) Aclar®, and composite sheets or films made from these materials, such as, for example, PVC/PVDC, PVC/HDPE, PVC/PCTFE, PP/COC (Polybar®) PVC/COC/PVDC, particularly suitable are PVC, PVDC, HDPE, PP, PET and composite sheets or films made from these, very particularly suitable PVC, PP, and PET. The plastic sheets or films can be employed as cavity sheet or film and/or as cover sheet or film. The cavity sheet or shaped film at least preferably consists of a plastic sheet or film.

Plastic sheets or films of low thickness are preferably and advantageously employed for the manufacture of the blister packs. The plastic sheets or films used as cavity sheet or film usually have a thickness of 10 to 500 μm, preferably 15 to 300 μm, particularly preferably 15 to 100 μm, very particularly preferably 15 to 50.

The blister pack may also include a frangible lidding, affixed to the shaped film, so that the single unit dose of the pharmaceutical product is substantially confined between the frangible lidding and the cavity. The frangible lidding is preferably made from aluminum foil sufficiently thin so as to enable a consumer to push the single unit dose through it by pressing on the underside of the cavity, or it may be made from an aluminum foil laminate (i.e., layers of aluminum, polyethylene terepthalate (PET) and/or paper) that is attached to the shaped film in a manner that permits the consumer to easily tear it away from each cavity, thereby gaining access to the single unit doses.

In an exemplary embodiment, the blister pack comprises a frangible lidding which is sealed or affixed to the shaped film such as by heat induction, for instance, so that the single unit doses of pharmaceutical product are substantially confined between the wells of the cavities and the frangible lidding.

A suitable blister pack, e.g. for a pharmaceutical composition or combination of the invention, comprises or is formed of a top foil or frangible layer (which is breachable by the tablets) and a bottom part (which contains pockets for the tablets). The top foil or frangible layer may contain a metallic foil, particularly an aluminium or aluminium alloy foil (e.g. having a thickness of 20 μm to 45 μm, preferably 20 μm to 25 μm) that is coated with a heat-sealing polymer layer on its inner side (sealing side). The bottom part may contain a multi-layer polymer foil (such as, e.g., poly(vinyl chloride) (PVC) coated with poly(vinylidene chloride) (PVDC); or a PVC foil laminated with poly(chlorotriflouroethylene) (PCTFE)) or a multi-layer polymer-metal-polymer foil (such as, e.g., a cold-formable laminated PVC/aluminium/polyamide composition).

Aluminium foil, which has low water permeability, is usually employed for sealing blister packs, such as in the frangible layer. Low water permeability is not necessary in the pack according to the invention, meaning that other materials can also be employed for sealing the blister packs. This enables the use of plastic sheets or films as cover sheet or film, where sheets or films made from the same material as the cavity sheet or film can also be used. Single-material packaging of this type is particularly advantageous since it can be recycled without prior separation of cavity sheet or film and cover sheet or film, which is particularly desired for environmental protection reasons. On use of plastic sheets or films as cover sheet or film, water vapour present in the cavities of the blister pack can also be removed through the cover sheet or film, which advantageously increases the drying rate of the pharmaceutical administration forms contained in the blister pack. If, in addition, a plastic sheet or film of very low material thickness is used, a further increase in the drying rate and easier removal of the solid pharmaceutical administration form contained in the blister pack arise, since this can be pushed through more easily, besides reduced material usage.

To ensure a long storage period especially under hot and wet climate conditions an additional overwrap or pouch made of a multi-layer polymer-metal-polymer foil (e.g. a laminated polyethylen/aluminium/polyester composition) may be used for the blister packs.

Blister cards can, for example, contain a foil backing as a barrier. Blister cards or packaging can include, for example, triplex blister film of different types, such as standard and high barrier films, including, for example, triplex Flexafarm Sbc (e.g., PVC 250 my+PE 25 my+PVDC 150 g/mq sbc grade) and Aquaba-PVC (e.g., PVC 250 my+AQUABA 160 g/mq), Aclar, Alu-Alu formats, triple layer blister foil (OPA) with soft tempered aluminium in central position, other layers PVC and polyamide, and new generation multilayer blister combined materials.

The article may further comprise a label or package insert, which refer to instructions customarily included in commercial packages of therapeutic products, that may contain information about the indications, usage, dosage, administration, contraindications and/or warnings concerning the use of such therapeutic products. In one embodiment, the label or package inserts indicates that the composition can be used for any of the purposes described herein.

For most situations, but not all, the blister pack will include a plurality of cavities, a plurality of airflow channels (at least one airflow channel per cavity), which together permit oxygen and moisture trapped in the plurality of cavities to easily pass out of the cavities, into the plurality of airflow channels and through the plurality of airflow channels to the reservoir comprising the absorbent. In some embodiments, however, there may even exist a plurality of airflow channels for every cavity in the blister.

Where there is a concern that the single unit doses of drugs inside the cavities in the blister pack may be too easily accessed by a child, embodiments of the invention may also include a hard plastic “shell pack” container, configured to receive, cover and protect the blister pack from direct access until the blister pack is extracted from inside the shell pack. In this alternative configuration, the blister pack may be inserted into the shell pack, and the shell pack sealed inside the sealed outer container during the package manufacturing stage.

### Moisture Absorbing Materials

A desiccant is any drying agent that removes moisture from the air. Desiccants include, but are not limited to, silica gel, clay desiccants, calcium sulfate, calcium chloride, calcium oxide, zeolite, activated alumina, activated charcoal and combinations thereof. However, other vapor or moisture absorbing mechanisms are not beyond the scope of the present invention. Other vapor or moisture absorbing materials include desiccants made from inorganic materials such a zeolites and aluminas. Such inorganic materials of vapour or moisture absorbing materials have high water absorption capacities and favourable water absorption isotherm shapes. The water absorption capacity of such materials typically varies from 20 to 50 weight percent. In the preferred embodiment, the absorbing material is a MINIPAX™ supplied by Multisorb Technologies in the United States and Silgelac in Europe (silica gel packaged inside TYVEK®, which is a nylon mesh bonded with a microporous polyurethane). Other exemplary moisture absorbing materials include, but are not limited to, alumina, bauxite, anhydrous, calcium sulphate, water-absorbing clay, activated bentonite clay, a molecular sieve, or other like materials which optionally include a moisture sensitive colour indicator such as cobalt chloride to indicate when the desiccant is no longer operable. While in the preferred embodiment of the present invention, the package is designed to substantially prevent ingression of water vapor and particulate matter into the enclosed volume, the moisture absorbing material is placed within the reservoir in order to absorb any residual moisture present in the atmosphere.

The desiccant should be present in an amount sufficient to absorb any residual moisture inside the package. Moreover, the desiccant should be present in an amount sufficient to absorb any moisture that possibly ingresses from the external environment. It is also possible to place the desiccant inside the reservoir.

Preferably the desiccant is selected from the group consisting of silica gel, zeolite, alumina, bauxite, anhydrous calcium sulphate, activated bentonite clay, water-absorbing clay, molecular sieve and any mixtures thereof.

Absorbents which may be present are in principle any type of desiccants, i.e. moisture-binding binders. Desiccants may include chemical substances which form hydrates with water. Examples of chemical substances of this type are anhydrous salts, which tend to absorb water or moisture and in the process form a stable hydrate. The moisture is bound and liberation thereof is prevented by a chemical reaction.

Desiccants may contain substances which are reactive. The substances react with water or moisture by forming a new substance. The newly formed substances are normally stable at low temperatures, which is only reversible with expenditure of high energy. Desiccants of this type are principally used for drying solvents and as water-absorbent material in the case of polymers which themselves have to remain in a reduced-moisture state.

Desiccants may bind the moisture by physical adsorption. The desiccant contains particles having fine capillaries into which the moisture is drawn. The pore size of the capillaries and the density thereof in the desiccant determine the absorption properties. Examples of desiccants of this type are molecular sieves, silica gels, certain synthetic polymers.

### Oxygen Absorber

In the pharmaceutical packaging systems described herein, oxygen absorbers absorb and remove oxygen from all components of the system. Oxygen absorbers are contemplated to be in any size or shape including sachet, pouch, capsule, label, strip, patch, cartridge, lining, sticker, etc., that is placed inside of the reservoir, but can also be integrated to the primary packaging. In some embodiments, the oxygen absorber is in the form of a capsule.

Suitable materials for oxygen absorbers include metal-based substances that remove oxygen by reacting with it by chemical bonding, generally forming a metal oxide component. Metal-based substances include elemental iron as well as iron oxide, iron hydroxide, iron carbide and the like. Other metals for use as oxygen absorbers include nickel, tin, copper and zinc. Metal-based oxygen absorbers are typically in the form of a powder to increase surface area. Powder formation of the metal-based oxygen absorbers is by any known method including, but not limited to, atomization, milling, pulverization, and electrolysis. Additional materials for oxygen absorbers include low molecular weight organic compounds such as ascorbic acid, sodium ascorbate, catechol and phenol, activated carbon and polymeric materials incorporating a resin and a catalyst. In some embodiments of the pharmaceutical packaging system, the oxygen absorber is a metal-based oxygen absorber. In certain instances of the pharmaceutical packaging system, the oxygen absorber is an iron-based oxygen absorber. In further instances of the pharmaceutical packaging system, the oxygen absorber is an iron-based oxygen absorber in the form of a canister.

Oxygen absorbents which can be used in the present invention include iron and glucose oxidase. A salt may be used as an electrolyte for oxidation of the iron. The iron may be hydrogen-reduced iron, electrolytically reduced iron, or chemically reduced iron. Although iron is preferred as the metallic oxygen absorbing agent, it will be appreciated that other metals may be used. These are, by way of example and not limitation, aluminum, copper, zinc, titanium, magnesium, and tin. Also, other elements which can be used in elemental or partially oxidized form are sodium, manganese, iodine, sulfur, and phosphorus.

The electrolytic salt may be sodium chloride or any other suitable food compatible salt including, but not limited to, sodium sulfate, potassium chloride, ammonium chloride, ammonium sulfate, calcium chloride, sodium phosphate, calcium phosphate, and magnesium chloride.

An example of a suitable thermoplastic resin containing an oxygen absorber is Amosorb™ 3000 (available from BP Amoco Chemicals). Other resins appropriate for the current invention include those made using ascorbic acid or other easily oxidized organic compounds.

### Coloring Agents

Coloring agents can be used to color code the absorbent, for example, to indicate that the absorbent is not to be ingested. Suitable coloring agents include, without limitation, natural and/or artificial compounds such as FD&C coloring agents, natural juice concentrates, pigments such as titanium oxide, silicon dioxide, iron oxides, zinc oxide, combinations thereof, and the like.

Colorants/opacifiers for use in the present invention include organic dyes and their lakes, inorganic colors and natural colors, including water soluble colors and water-insoluble colors (pigments). The compositions of the present invention can comprise from about 0% to about 10% by weight of the flavoring and/or coloring agent, preferably from about 0.1% to about 5%, and more preferably from about 2% to about 3%.

Pharmaceutical Products that are Sensitive to Oxygen and/or Moisture

In exemplary embodiments, the pharmaceutical product that is sensitive to oxygen and/or moisture is selected from the group consisting of morphine, hydromorphone, promethazine, dopamine, epinephrine, norepinephrine, esterified estrogen, ephedrine, pseudoephedrine, acetaminophen, ibuprofen, danofloxacin, erythromycin, penicillin, cyclosporine, methyldopate, cetirizine, diltiazem, verapamil, mexiletine, chlorothiazide, carbamazepine, selegiline, oxybutynin, vitamin A, vitamin B, vitamin C, L-cysteine, L-tryptophan, and combinations thereof.

Other Active Pharmaceutical Ingredients may include opioids. such as, for example, antitussive expectorants such as dihydrocodeine phosphate, codeine phosphate, and noscapine hydrochloride. In another embodiment, the opioid drugs are analgesics drugs such as hydrocodone, morphine, hydromorphone, oxycodone, codeine, levorphanol, meperidine, methadone, oxymorphone, buprenorphine, fentanyl and derivatives thereof, dipipanone, tramadol, etorphine, dihydroetorphine, butorphanol, levorphanol, or salts thereof or mixtures thereof. In one embodiment, the opioid is morphine, oxycodone, hydrocodone, or a salt thereof. In one embodiment, a morphine salt is morphine sulfate; an oxycodone salt is oxycodone HCl; and a codeine salt is codeine sulfate or phosphate.

Examples of specific active drug substances suitable for use in the pharmaceutical compositions provided herein include: anti-inflammatory and antirheumatic active drug substances, such as, for example: butylpyrazolidine, phenylbutazone, mofebutazone, oxyphenbutazone, clofezone, kebuzone, acetic acid derivatives and related substances, indometacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac, bufexamac, oxicam, piroxicam, tenoxicam, droxicam, lornoxicam, meloxicam, methotrexate, propionic acid derivatives, ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, fenamates, mefenamic acid, tolfenamic acid, flufenamic acid, meclofenamic acid, coxibs, celecoxib, rofecoxib, valdecoxib, parecoxib, etoricoxib, lumiracoxib, nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, feprazone, dipyrocetyl, acetylsalicylic acid, quinolines, oxycinchophen, gold preparations, sodium aurothiomalate, sodium aurotiosulfate, auranofin, aurothioglucose, aurotioprol, penicillamine or bucillamine.

In another embodiment, suitable active pharmaceutical ingredients can comprise analgesics, such as, for example: opioids, natural opium alkaloids, morphine, opium, hydromorphone, nicomorphine, oxycodone, dihydrocodeine, diamorphine, tapentadol, papavereturn, codeine, phenylpiperidine derivatives, ketobemidone, pethidine, fentanyl, diphenylpropylamine derivatives, dextromoramide, piritramide, dextropropoxyphene, bezitramide, methadone, benzomorphan derivatives, pentazocine, phenazocine, oripavine derivatives, buprenorphine, morphinan derivatives, butorphanol, nalbuphine, tilidine, tramadol, dezocine, salicylic acid and derivatives, acetylsalicylic acid, aloxiprin, choline salicylate, sodium salicylate, salicylamide, salsalate, ethenzamide, morpholine salicylate, dipyrocetyl, benorilate, diflunisal, potassium salicylate, guacetisal, carbasalate calcium, imidazole salicylate, pyrazolones, phenazone, metamizole sodium, aminophenazone, propyphenazone, nifenazone, anilides, paracetamol, phenacetin, bucetin, propacetamol, other analgesics and antipyretics, such as, for example: rimazolium, glafenine, floctafenine, viminol, nefopam, flupirtine, or ziconotide.

In another embodiment, suitable active pharmaceutical ingredients can comprise anaesthetics, such as, for example: ethers, diethyl ether, vinyl ether, halogenated hydrocarbons, halothane, chloroform, methoxyflurane, enflurane, trichloroethylene, isoflurane, desflurane, sevoflurane, barbiturates, methohexital, hexobarbital, thiopental, narcobarbital, opioid anaesthetics, fentanyl, alfentanil, sufentanil, phenoperidine, anileridine, remifentanil, other general anaesthetics, such as, for example: droperidol, ketamine, propanidid, alfaxalone, etomidate, propofol, hydroxybutyric acid, nitrous oxide, esketamine, xenon, esters of aminobenzoic acid, metabutethamine, procaine, tetracaine, chloroprocaine, benzocaine, amides, bupivacaine, lidocaine, mepivacaine, prilocaine, butanilicaine, cinchocaine, etidocaine, articaine, ropivacaine, levobupivacaine, esters of benzoic acid, cocaine, other local anaesthetics, such as, for example: ethyl chloride, dyclonine, phenol, or capsaicin.

In another embodiment, suitable active pharmaceutical ingredients can comprise antimigraine active drug substances, such as, for example: ergot alkaloids, dihydroergotamine, ergotamine, methysergide, lisuride, corticosteroid derivatives, flumedroxone, selective serotonin (SHT.sup.1) agonists, sumatriptan, naratriptan, zolmitriptan, rizatriptan, almotriptan, eletriptan, frovatriptan, other antimigraine preparations, pizotifen, clonidine, iprazochrome, dimetotiazine, or oxetorone.

In another embodiment, suitable active pharmaceutical ingredients can comprise antiepileptic active drug substances, such as, for example: barbiturates and derivatives, methylphenobarbital, phenobarbital, primidone, barbexaclone, metharbital, hydantoin derivatives, ethotoin, phenytoin, amino(diphenylhydantoin) valeric acid, mephenytoin, fosphenytoin, oxazolidine derivatives, paramethadione, trimethadione, ethadione, succinimide derivatives, ethosuximide, phensuximide, mesuximide, benzodiazepine derivatives, clonazepam, carboxamide derivatives, carbamazepine, oxcarbazepine, rufinamide, fatty acid derivatives, valproic acid, valpromide, aminobutyric acid, vigabatrin, progabide, tiagabine, other antiepileptics, such as, for example: sultiame, phenacemide, lamotrigine, felbamate, topiramate, gabapentin, pheneturide, levetiracetam, zonisamide, pregabalin, stiripentol, lacosamide, or beclamide.

In another embodiment, suitable active pharmaceutical ingredients can comprise anticholinergic active drug substances, such as, for example: tertiary amines, trihexyphenidyl, biperiden, metixene, procyclidine, profenamine, dexetimide, phenglutarimide, mazaticol, bornaprine, tropatepine, ethers chemically close to antihistamines, etanautine, orphenadrine (chloride), ethers of tropine or tropine derivatives, benzatropine, or etybenzatropine.

In another embodiment, suitable active pharmaceutical ingredients can comprise dopaminergic active drug substances, such as, for example: dopa and dopa derivatives, levodopa, melevodopa, etilevodopa, adamantane derivatives, amantadine, dopamine agonists, bromocriptine, pergolide, dihydroergocryptine mesylate, ropinirole, pramipexole, cabergoline, apomorphine, piribedil, rotigotine, monoamine, oxidase B inhibitors, selegiline, rasagiline, other dopaminergic agents, such as, for example: tolcapone, entacapone, or budipine.

In another embodiment, suitable active pharmaceutical ingredients can comprise antipsychotic active drug substances, such as, for example: phenothiazines with aliphatic side-chain, chlorpromazine, levomepromazine, promazine, acepromazine, triflupromazine, cyamemazine, chlorproethazine, phenothiazines with piperazine structure, dixyrazine, fluphenazine, perphenazine, prochlorperazine, thiopropazate, trifluoperazine, acetophenazine, thioproperazine, butaperazine, perazine, phenothiazines with piperidine structure, periciazine, thioridazine, mesoridazine, pipotiazine, butyrophenone derivatives, haloperidol, trifluperidol, melperone, moperone, pipamperone, bromperidol, benperidol, droperidol, fluanisone, indole derivatives, oxypertine, molindone, sertindole, ziprasidone, thioxanthene derivatives, flupentixol, clopenthixol, chlorprothixene, tiotixene, zuclopenthixol, diphenylbutylpiperidine derivatives, fluspirilene, pimozide, penfluridol, diazepines, oxazepines, thiazepines, loxapine, clozapine, olanzapine, quetiapine, neuroleptics, tetrabenazine, benzamides, sulpiride, sultopride, tiapride, remoxipride, amisulpride, veralipride, levosulpiride, lithium, other antipsychotics, such as, for example prothipendyl, risperidone, clotiapine, mosapramine, zotepine, aripiprazole, or paliperidone.

In another embodiment, suitable active pharmaceutical ingredients can comprise anxiolytic active drug substances, such as, for example: benzodiazepine derivatives, diazepam, chlordiazepoxide, medazepam, oxazepam, potassium clorazepate, lorazepam, adinazolam, bromazepam, clobazam, ketazolam, prazepam, alprazolam, halazepam, pinazepam, camazepam, nordazepam, fludiazepam, ethyl loflazepate, etizolam, clotiazepam, cloxazolam, tofisopam, diphenylmethane derivatives, hydroxyzine, captodiame, carbamates, meprobamate, emylcamate, mebutamate, dibenzo-bicyclo-octadiene derivatives, benzoctamine, azaspirodecanedione derivatives, buspirone, other anxiolytics, such as, for example: mephenoxalone, gedocarnil, or etifoxine.

In another embodiment, suitable active pharmaceutical ingredients can comprise hypnotic and sedative active drug substances, such as, for example: barbiturates, pentobarbital, amobarbital, butobarbital, barbital, aprobarbital, secobarbital, talbutal, vinylbital, vinbarbital, cyclobarbital, heptabarbital, reposal, methohexital, hexobarbital, thiopental, ethallobarbital, allobarbital, proxibarbal, aldehydes and derivatives, chloral hydrate, chloralodol, acetylglycinamide chloral hydrate, dichloralphenazone, paraldehyde, benzodiazepine emepronium derivatives, flurazepam, nitrazepam, flunitrazepam, estazolam, triazolam, lormetazepam, temazepam, midazolam, brotizolam, quazepam, loprazolam, doxefazepam, cinolazepam, piperidinedione derivatives, glutethimide, methyprylon, pyrithyldione, benzodiazepine related drugs, zopiclone, zolpidem, zaleplon, ramelteon, other hypnotics and sedatives, such as, for example: methaqualone, clomethiazole, bromisoval, carbromal, scopolamine, propiomazine, triclofos, ethchlorvynol, valerian, hexapropymate, bromides, apronal, valnoctamide, methylpentynol, niaprazine, melatonin, dexmedetomidine, or dipiperonylaminoethanol.

In another embodiment, suitable active pharmaceutical ingredients can comprise antidepressant active drug substances, such as, for example: non-selective monoamine reuptake inhibitors, desipramine, imipramine, imipramine oxide, clomipramine, opipramol, trimipramine, lofepramine, dibenzepin, amitriptyline, nortriptyline, protriptyline, doxepin, iprindole, melitracen, butriptyline, dosulepin, amoxapine, dimetacrine, amineptine, maprotiline, quinupramine, selective serotonin reuptake inhibitors, zimeldine, fluoxetine, citalopram, paroxetine, sertraline, alaproclate, fluvoxamine, etoperidone, escitalopram, monoamine oxidase inhibitors, isocarboxazid, nialamide, phenelzine, tranylcypromine, iproniazide, iproclozide, monoamine oxidase A inhibitors, moclobemide, toloxatone, other antidepressants, such as, for example: oxitriptan, tryptophan, mianserin, nomifensine, trazodone, nefazodone, minaprine, bifemelane, viloxazine, oxaflozane, mirtazapine, medifoxamine, tianeptine, pivagabine, venlafaxine, milnacipran, reboxetine, gepirone, duloxetine, agomelatine, desvenlafaxine, centrally acting sympathomimetics, such as, for example: amfetamine, dexamfetamine, lisdexamfetamine, metamfetamine, methylphenidate, dexmethylphenidate, pemoline, fencamfamin, modafinil, fenozolone, atomoxetine, fenetylline, xanthine derivatives, caffeine, propentofylline, other psychostimulants and nootropics, such as, for example meclofenoxate, pyritinol, piracetam, deanol, fipexide, citicoline, oxiracetam, pirisudanol, linopirdine, nizofenone, aniracetam, acetylcarnitine, idebenone, prolintane, pipradrol, pramiracetam, adrafinil, or vinpocetine.

In another embodiment, suitable active pharmaceutical ingredients can comprise anti-dementia active drug substances, such as, for example: anticholinesterases, tacrine, donepezil, rivastigmine, galantamine, other anti-dementia drugs, memantine, or ginkgo biloba.

In another embodiment, suitable active pharmaceutical ingredients can comprise other nervous system active drug substances, such as, for example: parasympathomimetics, anticholinesterases, neostigmine, pyridostigmine, distigmine, ambenonium, choline esters, carbachol, bethanechol, and other parasympathomimetics, such as, for example, pilocarpine, or choline alfoscerate.

Active drug substances used in addictive disorders, such as, for example: nicotine, bupropion, varenicline, disulfiram, calcium carbimide, acamprosate, naltrexone, buprenorphine, methadone, levacetylmethadol, lofexidine, betahistine, cinnarizine, flunarizine, acetylleucine, gangliosides and ganglioside derivatives, tirilazad, riluzole, xaliproden, hydroxybutyric acid, or amifampridine.

In another embodiment, suitable active pharmaceutical ingredients can comprise opium alkaloids and derivatives, such as, for example: ethylmorphine, hydrocodone, codeine, opium alkaloids with morphine, normethadone, noscapine, pholcodine, dextromethorphan, thebacon, dimemorfan, acetyldihydrocodeine, benzonatate, benproperine, clobutinol, isoaminile, pentoxyverine, oxolamine, oxeladin, clofedanol, pipazetate, bibenzonium bromide, butamirate, fedrilate, zipeprol, dibunate, droxypropine, prenoxdiazine, dropropizine, cloperastine, meprotixol, piperidione, tipepidine, morclofone, nepinalone, levodropropizine, or dimethoxanate.

In another embodiment, the active pharmaceutical ingredient may be a substance with abuse potential that presents a safety risk. Such active drug substance may include: 1-(1-phenylcyclohexyl)pyrrolidine, 1-(2-phenylethyl)-4-phenyl-4-acetoxypiperidine, 1-[1-(2-thienyl)-cyclohexylpiperidine, 1-[1-(2-thienyl)cyclohexyl]pyrrolidine, 1-methyl-4-phenyl-4-propionoxy-piperidine, 1-phenylcyclohexylamine, 1-piperidinocyclohexanecarbonitrile, 2,5-dimethoxy-4-ethylamphetamine, 2,5-dimethoxyamphetamine, 2C—B-(4-bromo-2,5-dimethoxyl)enethylamine), 2C-D (2,5-dimethoxy-4-methylphenethylamine), 2C—I(4-iodo-2,5-dimethoxy-phenethylamine), 2C-T-2 (2,5-dimethoxy-4-ethylthiophenethylamine), 2C-T-4 (2,5-dimethoxy-4-isopropyl thiophenethylamine), 2C-T-7 (2,5-dimethoxy-4-(n)-propylthiophenethylamine), 3,4-methylene-dioxymethamphetamine, 3,4,5-trimethoxyamphetamine, 3,4-methylenedioxyamphetamine, 3,4-methylenedioxy-N-ethylamphetamine, 3-methylfentanyl, 3-methylthiofentanyl, 4-brorno-2,5-dimethoxyamphetamine, 4-bromo-2,5-dimethoxyphenethylamine, 4-methoxyamphetamine, 4-methyl-2,5-dimethoxyamphetamine, 4-methylaminorex (cis isomer), 5-MeO-DIPT (5-methoxy-N,N-diisopropyltryptamine), 5-MeO-DMT (5-methoxy-N,N-dimethyltryptamine), 5-methoxy-3,4-methylenedioxyamphetamine, acetorphine, acetorphine, acetyl-alpha-methylfentanyl, acetyl-alpha-methylfentanyl, acetyldihydrocodeine, acetylmethadol, acetylmethadol, alfentanil, allobarbital, allylprodine, alphacetylmethadol except levo-alphacetylmethadol, alpha-ethyltryptamine, alphameprodine, alphamethadol, alphamethadol, alpha-methylfentanyl, alpha-methylthiofentanyl, alphaprodine, alprazolam, amfepramon, amfetaminil, amineptin, aminorex, amobarbital, amphetamine, dextroamphetamine, amilnitrite (all isomers of the amyl group), anabolic steroids, anileridine, aprobarbital, barbital, barbituric acid derivative, BDB (3,4-methylenedioxyphenyl)-2-butanamine), benzethidin, benzethidine, benzoylecgonine, benzphetamine, benzphetamine, benzylmethylcetone, benzylmorphine, betacetylmethadol, beta-hydroxy-3-methylfentanyl, beta-hydroxyfentanyl, betameprodine, betameprodine, betamethadol, betaprodine, bezitramide, bezitramide, boldenone, brolamfetamine, bromazepam, brotizolam, bufotenine, buprenorphine, butabarbital, butalbital, butobarbital, butorphanol, BZP (A2)(1-benzylpiperazin), camazepam, cannabis, carfentanil, catha edulis, cathine, cathinone, chloral betaine, chloral hydrate, chlordiazepoxide, chlorhexadol, chlorotestosterone (same as clostebol), chlorphentermine, clobazam, clonazepam, clonitazene, clonitazene, clorazepate, clortermine, clostebol, clotiazepam, cloxazolam, coca leaves, cocaine, codeine, codeine and isoquinoline alkaloid, codeine methylbromide, codeine-N-oxide, codoxime, cyclobarbital (hexemal NFN), cyprenorphine, dehydrochlormethyltestosterone, delorazepam, desomorphine, dexamfetamine, dexfenfluramine, dexmethylphenidate, dextromoramide, dextropropoxyphene, diacetylmorphine, diampromide, diazepam, dichloralphenazone, diethylpropion, diethylthiambutene, diethyltryptamine, difenoxin, dihydrocodeine, dihydroetorphine, dihydromorphine, dihydrotestosterone, dimenoxadol, dimepheptanol, dimethylthiambutene, dimethyltryptamine, dioxaphetyl butyrate, diphenoxylate, dipipanone, diprenorphine, dronabinol, drostanolone, drotebanol, ecgonine, estazolam, ethchlorvynol, ethinamate, ethyl loflazepate, ethylestrenol, ethylmethylthiambutene, ethylmorphine, ethylmorphine, eticyclidine, etilamfetamine, etonitazene, etorphine, etoxeridine, etryptamine, fencamfamin, fenethylline, fenetylline, fenfluramine, fenproporex, fentanyl, fludiazepam, flunitrazepam, fluoxymesterone, flurazepam, formebolone, fungi and spores of the species psilocybe semilanceata, furethidine, gamma hydroxybutyric acid, glutethimide, halazepam, haloxazolam, heroine, hydrocodone, hydrocodone & isoquinoline alkaloid, hydromorphinol, hydromorphone, hydroxypethidine, ibogaine, isobutyl nitrite, isomethadone, ketamine, ketazolam, ketobemidone, levamfetamine, levo-alphacetylmethadol, levo-methamphetamine, levomethorphan, levomoramide, levophenacylmorphan, levorphanol, lisdexamfetamine, loprazolam, lorazepam, lormetazepam, lysergic acid, lysergic acid amide, lysergic acid diethylamide, marijuana, mazindol, MBDN (N-methyl-1-(3,4-methylenedioxyphenyl)-2-butanamine), mCPP (1-(3-chlorphenyl)piperazine), mebutamate, mecloqualone, medazepam, mefenorex, MeOPP (1-(4-methoxyphenyl)piperazine), meperidine, meperidine intermediate, meprobamate, mescaline, mesocarb, mesterolone, metamfetamine, metazocine, methadone, methadone intermediate, methamphetamine, methandienone, methandrolone, methandriol, methandrostenolone, methaqualone, methcathinone, methenolone, methohexital, methyldesorphine, methyldihydromorphine, methylphenidate, methylphenobarbital (mephobarbital), methyltestosterone, methyprylone, metopone, mibolerone, midazolam, modafinil, moramide-intermediate, morpheridine, morphine, morphine methylbromide, morphine methylsulfonate, morphine-N-oxide, myrophine, N,N-dimethylamphetamine, nabilone, nalorphine, nandrolone, N-ethyl-1-phenylcyclohexylamine, N-ethyl-3-piperidyl benzilate, N-ethylamphetamine, N-hydroxy-3,4-methylenedioxyamphetamine, nicocodeine, nicocodine, nicodicodine, nicomorphine, nimetazepam, nitrazepam, N-methyl-3-piperidyl benzilate, noracymethadol, norcodeine, nordiazepam, norethandrolone, norlevorphanol, normethadone, normorphine, norpipanone, norpipanone, opium, oxandrolone, oxazepam, oxazolam, oxycodone, oxymesterone, oxymetholone, oxymorphone, para-fluorofentanyl, parahexyl, paraldehyde, pemoline, pentazocine, pentobarbital, petrichloral, peyote, phenadoxone, phenampromide, phenazocine, phencyclidine, phendimetrazine, phenmetrazine, phenobarbital, phenomorphan, phenoperidine, phentermine, phenylacetone, pholcodine, piminodine, pinazepam, pipradrole, piritramide, PMMA (paramethyxymethyl amphetamine), prazepam, proheptazine, properidine, propiram, psilocybine, psilocine, pyrovalerone, quazepam, racemethorphane, racemoramide, racemorphane, remifentanil, salvia divinorum, salvinorin A, secobarbital, secobarbital, sibutramine, SPA, stanolone, stanozolol, sufentanil, sulfondiethylmethane, sulfonethylmethane, sulfonmethane, talbutal, temazepam, tenamfetamine, testolactone, testosterone, tetrahydrocannabinols, tetrazepam, TFMPP (1-(3-triflourmethylphenyl)piperazine), thebacon, thebaine, thiamylal, thiofentanyl, thiopental, tiletamine and zolazepam in combination, tilidine, trenbolone, triazolam, trimeperidine, vinbarbital, zaleplon, zipeprol, zolpidem, or zopiclone.

Other suitable examples of active drug substances include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, dextropropoxyphene, ketobemidone, levallorphan, levorphanol, levophenacylmorphan, lofentanil, meperidine, meptazinol, metazocine, methadone, metopon, morphine, morphine 6-glucuronide, morphine 3-glucuronide, myrophine, nalbuphine, narcine, nicomorphine, norlevorphanol, normethadone, nalorphine, normorphine, norpipanone, opium, oxycodone, oxycodeine, oxymorphone, papavereturn, pentazocine, phenadoxone, phenomorphan, phenazocine, phenoperidine, piminodine, piritramide, propheptazine, promedol, properidine, propiram, propoxyphene, sufentanil, tilidine, tramadol, thebaine, levo-alphacetylmethadol (LAAM), remifentanil, carfentanyl, ohmefentanyl, MPPP, prodine, PEPAP, levomethorphan, etorphine, lefetamine, loperamide, diphenoxylate, or pethidine.

Other examples of active drug substances suitable for use in the pharmaceutical compositions described herein include anabolic steroids, cannabis, cocaine, or diazepam.

In another embodiment, the active drug substance comprises the therapeutic classes including non-steroidal anti-inflammatory substances or antirheumatic active drug substances. In other embodiments, the active drug substance comprises analgesics, opioids, antipyretics, anaesthetics, antimigraine agents, antiepileptics, anti-parkinson agents, dopaminergic agents, antipsychotics, anxiolytics, sedatives, antidepressants, psychostimulants agents, dopamine, noradrenaline, nicotinic, alfa-adrenergic, serotonin, H3 antagonists used for ADHD or nootropics agents used in addictive disorders.

In other embodiments, the active drug substance comprises therapeutic classes including anaesthetics, centrally acting analgesics, sedative-hypnotics, anxiolytics, appetite suppressants, decongestants, antitussives, antihistamines, antiemetics, antidiarrheals, and drugs used to treat narcolepsy, or attention deficit hyperactivity disorder.

In another embodiment, the active drug substance is associated with abuse syndromes and the active drug substance may, for example, be selected from opioids, CNS depressants, CNS stimulants, cannabinoids, nicotine-like compounds, glutamate antagonists, or N-methyl-D-aspartate (NMDA) antagonists.

In another embodiment, the active drug substance is an analgesic. Examples of analgesics suitable for use in the pharmaceutical compositions described herein include, for example, opioids, natural opium alkaloids, morphine, opium, hydromorphone, nicomorphine, oxycodone, dihydrocodeine, diamorphine, tapentadol, papaveretum, codeine, phenylpiperidine derivatives, ketobemidone, pethidine, fentanyl, diphenylpropylamine derivatives, dextromoramide, piritramide, dextropropoxyphene, bezitramide, methadone, benzomorphan derivatives, pentazocine, phenazocine, oripavine derivatives, buprenorphine, morphinan derivatives, butorphanol, nalbuphine, tilidine, tramadol, dezocine, salicylic acid and derivatives, acetylsalicylic acid, aloxiprin, choline salicylate, sodium salicylate, salicylamide, salsalate, ethenzamide, morpholine salicylate, dipyrocetyl, benorilate, diflunisal, potassium salicylate, guacetisal, carbasalate calcium, imidazole salicylate, pyrazolones, phenazone, metamizole sodium, aminophenazone, propyphenazone, nifenazone, anilides, paracetamol, phenacetin, bucetin, propacetamol, other analgesics and antipyretics such as, for example, rimazolium, glafenine, floctafenine, viminol, nefopam, flupirtine, or ziconotide.

In another embodiment, the active drug substance is an opioid. Where an opioid is included as an active drug substance, the opioid may comprise naturally occurring opioids, synthetic opioids, or semisynthetic opioids.

In other embodiment, the active drug substance comprises amfetamine, dexamfetamine, lisdexamfetamine, metamfetamine, methylphenidate, dexmethylphenidate, or combinations thereof.

Where an opioid is used as an active drug substance, the opioid may be present in any of its crystalline, polymorphous, semi-crystalline, and amorphous or polyamorphous forms. Furthermore, in another embodiment, an opioid used as an active drug substance may be present in one or more forms selected from its crystalline, polymorphous, semi-crystalline, or amorphous or polymorphous forms.

### Manufacturing the Package

The blister packs of the present invention may be manufacture using an automatic blister thermoformer known in the art. In exemplary embodiments, the blister thermoformer forms the cavities, reservoirs, and the airflow channels, fills the cavities with tablets/caplets and the reservoir with an absorber, covers and seals the blister pack with the frangible lidding, and then die-cuts the sealed blister packs into their desired final marketing configuration. The sealed blister packs may then be transported to either an automatic or manual pouch machine.

The invention will be illustrated in more detail with reference to the following Examples, but it should be understood that the present invention is not deemed to be limited thereto.

### EXAMPLES

### Example 1

Dronabinol Capsules, 2.5 mg

| S. | Name of Packaging | | Qty./36000 |
|---|---|---|---|
| No. | Component | Unit | units |
| 1. | Dronabinol Capsules, 2.5 mg | No | 36000.00 |
| 2. | Aquaba 200-114 mm (PVC Film) | Kgs | 40.00 |
| 3. | CrisPak PL-108 mm (PVC Film) | Kgs | 40.00 |
| 4. | 1 gram Sorb-it Canister | No. | 9000.00 |
| 5. | Display Carton (8.686 × 3.625 × 4.4063) | Nos | 120.00 |
| 6. | Display Carton Label for | Nos | 120.00 |
| 7. | 200/225 cc Master Carton | Nos | 30.00 |

### Example 2

Dronabinol Capsules, 2.5 mg

| S. | Name of Packaging | | Qty./36000 |
|---|---|---|---|
| No. | Component | Unit | units |
| 1. | Dronabinol Capsules, 2.5 mg | No | 36000.00 |
| 2. | Aquaba 200-114 mm (PVC Film) | Kgs | 40.00 |
| 3. | CrisPak PL-108 mm (PVC Film) | Kgs | 40.00 |
| 4. | 2 gram Sorb-it Tyvek (22 × 53) | No. | 9000.00 |
| 5. | Display Carton (8.686 × 3.625 × 4.4063) | Nos | 120.00 |
| 6. | Display Carton Label for | Nos | 120.00 |
| 7. | 200/225 cc Master Carton | Nos | 30.00 |

### Example 3

Dronabinol Capsules, 2.5 mg

| S. | Name of Packaging | | Qty./36000 |
|---|---|---|---|
| No. | Component | Unit | units |
| 1. | Dronabinol Capsules, 2.5 mg | No | 36000.00 |
| 2. | Aquaba 200-114 mm (PVC Film) | Kgs | 40.00 |
| 3. | CrisPak PL-108 mm (PVC Film) | Kgs | 40.00 |
| 4. | StabilOX ® DF-100-H31 (Tyvek) | No. | 9000.00 |
| 5. | Display Carton (8.686 × 3.625 × 4.4063) | Nos | 120.00 |
| 6. | Display Carton Label for | Nos | 120.00 |
| 7. | 200/225 cc Master Carton | Nos | 30.00 |

### Example 4

Dronabinol Capsules, 2.5 mg

| S. | Name of Packaging | | Qty./36000 |
|---|---|---|---|
| No. | Component | Unit | units |
| 1. | Dronabinol Capsules, 2.5 mg | No | 36000.00 |
| 2. | Aquaba 200-114 mm (PVC Film) | Kgs | 40.00 |
| 3. | CrisPak PL-108 mm (PVC Film) | Kgs | 40.00 |
| 4. | StabilOX ® DF-200-H31 (Tyvek) | No. | 9000.00 |
| 5. | Display Carton (8.686 × 3.625 × 4.4063) | Nos | 120.00 |
| 6. | Display Carton Label for | Nos | 120.00 |
| 7. | 200/225 cc Master Carton | Nos | 30.00 |

### Example 5

Dronabinol Capsules, 10 mg

| S. | Name of Packaging | | Qty./36000 |
|---|---|---|---|
| No. | Component | Unit | units |
| 1. | Dronabinol Capsules, 10 mg | No | 36000.00 |
| 2. | Aquaba 200-114 mm (PVC Film) | Kgs | 40.00 |
| 3. | CrisPak PL-108 mm (PVC Film) | Kgs | 40.00 |
| 4. | 1 gram Sorb-it Canister | No. | 9000.00 |
| 5. | Display Carton (8.686 × 3.625 × 4.4063) | Nos | 120.00 |
| 6. | Display Carton Label for | Nos | 120.00 |
| 7. | 200/225 cc Master Carton | Nos | 30.00 |

### Example 6

Dronabinol Capsules, 10 mg

| S. | Name of Packaging | | Qty./36000 |
|---|---|---|---|
| No. | Component | Unit | units |
| 1. | Dronabinol Capsules, 10 mg | No | 36000.00 |
| 2. | Aquaba 200-114 mm (PVC Film) | Kgs | 40.00 |
| 3. | CrisPak PL-108 mm (PVC Film) | Kgs | 40.00 |
| 4. | 2 gram Sorb-it Tyvek (22 × 53) | No. | 9000.00 |
| 5. | Display Carton (8.686 × 3.625 × 4.4063) | Nos | 120.00 |
| 6. | Display Carton Label for | Nos | 120.00 |
| 7. | 200/225 cc Master Carton | Nos | 30.00 |

### Example 7

Dronabinol Capsules, 10 mg

| S. | Name of Packaging | | Qty./36000 |
|---|---|---|---|
| No. | Component | Unit | units |
| 1. | Dronabinol Capsules, 10 mg | No | 36000.00 |
| 2. | Aquaba 200-114 mm (PVC Film) | Kgs | 40.00 |
| 3. | CrisPak PL-108 mm (PVC Film) | Kgs | 40.00 |
| 4. | StabilOX ® DF-100-H31 (Tyvek) | No. | 9000.00 |
| 5. | Display Carton (8.686 × 3.625 × 4.4063) | Nos | 120.00 |
| 6. | Display Carton Label for | Nos | 120.00 |
| 7. | 200/225 cc Master Carton | Nos | 30.00 |

### Example 8

Dronabinol Capsules, 10 mg

| S. | Name of Packaging | | Qty./36000 |
|---|---|---|---|
| No. | Component | Unit | units |
| 1. | Dronabinol Capsules, 10 mg | No | 36000.00 |
| 2. | Aquaba 200-114 mm (PVC Film) | Kgs | 40.00 |
| 3. | CrisPak PL-108 mm (PVC Film) | Kgs | 40.00 |
| 4. | StabilOX ® DF-200-H31 (Tyvek) | No. | 9000.00 |
| 5. | Display Carton (8.686 × 3.625 × 4.4063) | Nos | 120.00 |
| 6. | Display Carton Label for | Nos | 120.00 |
| 7. | 200/225 cc Master Carton | Nos | 30.00 |

### Example 9

Pravastatin Sodium Tablets, USP 20 mg

| S. | Name of Packaging | | Qty./10000 |
|---|---|---|---|
| No. | Component | Unit | units |
| 1. | Pravastatin Sodium Tablets, USP 20 mg | No | 10000.00 |
| 2. | Aquaba 200-114 mm (PVC Film) | Kgs | 15.00 |
| 3. | CrisPak PL-108 mm (PVC Film) | Kgs | 15.00 |
| 4. | 2 gram Sorb-it Canister | No. | 1000.00 |
| 5. | Display Carton (8.686 × 3.625 × 4.4063) | Nos | 100.00 |
| 6. | Display Carton Label for | Nos | 100.00 |
| 7. | 200/225 cc Master Carton | Nos | 20.00 |

### Example 10

Pravastatin Sodium Tablets, USP 20 mg

| S. | Name of Packaging | | Qty./10000 |
|---|---|---|---|
| No. | Component | Unit | units |
| 1. | Pravastatin Sodium Tablets, USP 20 mg | No | 10000.00 |
| 2. | Aquaba 200-114 mm (PVC Film) | Kgs | 15.00 |
| 3. | CrisPak PL-108 mm (PVC Film) | Kgs | 15.00 |
| 4. | 3 gram Sorb-it Tyvek (22 × 53) | No. | 1000.00 |
| 5. | Display Carton (8.686 × 3.625 × 4.4063) | Nos | 100.00 |
| 6. | Display Carton Label for | Nos | 100.00 |
| 7. | 200/225 cc Master Carton | Nos | 20.00 |

### Example 11

Pravastatin Sodium Tablets, USP 20 mg

| S. | Name of Packaging | | Qty./10000 |
|---|---|---|---|
| No. | Component | Unit | units |
| 1. | Pravastatin Sodium Tablets, USP 20 mg | No | 10000.00 |
| 2. | Aquaba 200-114 mm (PVC Film) | Kgs | 15.00 |
| 3. | CrisPak PL-108 mm (PVC Film) | Kgs | 15.00 |
| 4. | StabilOX ® DF-200-H31 (Tyvek) | No. | 1000.00 |
| 5. | Display Carton (8.686 × 3.625 × 4.4063) | Nos | 100.00 |
| 6. | Display Carton Label for | Nos | 100.00 |
| 7. | 200/225 cc Master Carton | Nos | 20.00 |

### Example 12

Pravastatin Sodium Tablets, USP 20 mg

| S. | Name of Packaging | | Qty./10000 |
|---|---|---|---|
| No. | Component | Unit | units |
| 1. | Pravastatin Sodium Tablets, USP 20 mg | No | 10000.00 |
| 2. | Aquaba 200-114 mm (PVC Film) | Kgs | 15.00 |
| 3. | CrisPak PL-108 mm (PVC Film) | Kgs | 15.00 |
| 4. | StabilOX ® DF-300-H31 (Tyvek) | No. | 1000.00 |
| 5. | Display Carton (8.686 × 3.625 × 4.4063) | Nos | 100.00 |
| 6. | Display Carton Label for | Nos | 100.00 |
| 7. | 200/225 cc Master Carton | Nos | 20.00 |

While the invention has been described in detail and with reference to specific examples thereof, it will be apparent to one skilled in the art that various changes and modifications can be made therein without departing from the spirit and scope thereof.

What is claimed is:

1. A packaging system for a pharmaceutical product that is sensitive to oxygen and/or moisture comprising:
a blister pack comprising:
(i) a blister sheet comprising of an array of interconnected cavities connected through channels that allows the exchange of air between them;
(ii) wherein one or more of the cavities serve as a reservoirs holding an oxygen scavenger and/or a desiccant at concentrations high enough to provide higher equilibration time for oxygen and/or moisture;
(iii) further wherein one or more of the cavities comprise at least one pharmaceutical product that is sensitive to oxygen and/or moisture; and
(iii) a frangible lidding sealed to the sheet protecting the product in the cavity.

2. The package in claim 1, wherein the pharmaceutical product contains an active that comprises at least one amine as a part of chemical groups.

3. The package in claim 1, where in the pharmaceutical products is an active that is selected from the group consisting of cannabinoids, drobinol, dronabinol, δ-tetrahydrocannabinol, cannabidiol, and combinations thereof.

4. The package in claim 1, wherein the pharmaceutical product is selected from the group consisting of statins, lovastatin, simvastatin, fluvastatin, atorvastatin, pitavastatin, and combinations thereof.

5. The package in claim 1, wherein the pharmaceutical product is selected from the group consisting of morphine, hydromorphone, promethazine, dopamine, epinephrine, norepinephrine, esterified estrogen, ephedrine, pseudoephedrine, acetaminophen, ibuprofen, danofloxacin, erythromycin, penicillin, cyclosporine, methyldopate, cetirizine, diltiazem, verapamil, mexiletine, chlorothiazide, carbamazepine, selegiline, oxybutynin, vitamin A, vitamin B, vitamin C, L-cysteine, L-tryptophan, and combinations thereof.

6. The package in claim 1 wherein the blister sheet that has light and moisture protectant properties.

7. The package in claim 1 wherein the blister sheet is made of materials selected from the group consisting of polyvinylchloride, polyvinylidene chloride, polycarbonate, polyester, copolyester, acrylonitrile, low density polyethylene, polypropylene, and combinations thereof.

8. The package in claim 1 wherein the oxygen scavenger is selected from the group consisting of organic, inorganic, metallic, non-metallic, and enzymatic in nature.

9. The package in claim 1, where in the desiccants is selected from the group consisting of organic desiccants, inorganic desiccants, uncoated polymers, coated adsorbent polymers, and combinations thereof.

10. The package in claim 1 wherein the frangible lidding has moisture barrier properties.

11. A blister package for an oxygen and/or moisture sensitive pharmaceutical product comprising:
(i) a shaped film comprising:
(A) a plurality of cavities configured to each hold a single unit dose of the pharmaceutical product, and
(B) at least one airflow channel, and
(C) at least one reservoir;
wherein the at least one airflow channel connects each of the plurality of cavities to the at least one reservoir;
(ii) a frangible lidding sealed to the shaped film so that the unit dose is substantially confined between said frangible lidding and said at least one cavity;
(iii) an absorbent confined between the reservoir and the frangible lidding;
(iii) optionally a backing film; and
(iv) single unit doses of an oxygen and/or moisture sensitive pharmaceutical product confined between the cavities and the frangible lidding.

12. The package of claim 11, wherein the absorbent comprises a desiccant and an oxygen absorber, wherein the desiccant is selected from the group consisting of silica gel, clay desiccants, calcium sulfate, calcium chloride, calcium oxide, zeolite, activated alumina, activated charcoal, and combinations thereof, and the oxygen absorber is selected from the group consisting of iron oxidase, glucose oxidase, sodium sulfate, potassium chloride, ammonium chloride, ammonium sulfate, calcium chloride, sodium phosphate, calcium phosphate, magnesium chloride, and combinations thereof.

13. The package of claim 12, wherein the absorbent comprises a moisture sensitive color indicator to indicate when the desiccant is no longer operable.

14. The package of claim 11, wherein the cavities are interconnected by airflow channels.

15. The package of claim 11, wherein the pharmaceutical product is selected from the group consisting of morphine, hydromorphone, promethazine, dopamine, epinephrine, norepinephrine, esterified estrogen, ephedrine, pseudoephedrine, acetaminophen, ibuprofen, danofloxacin, erythromycin, penicillin, cyclosporine, methyldopate, cetirizine, diltiazem, verapamil, mexiletine, chlorothiazide, carbamazepine, selegiline, oxybutynin, vitamin A, vitamin B, vitamin C, L-cysteine, L-tryptophan, and combinations thereof.

16. The package of claim 11, wherein the shaped film comprises polyvinyl chloride, polyvinylidene chloride, polycarbonate, polyester, copolyester, acrylonitrile, low density polyethylene, polypropylene, or a combination thereof.

17. The package of claim 11, wherein an active pharmaceutical ingredient in the pharmaceutical product is an active that is selected from the group consisting of cannabinoids, drobinol, dronabinol, δ-tetrahydrocannabinol, cannabidiol, statins, lovastatin, simvastatin, fluvastatin, atorvastatin, pitavastatin, and combinations thereof.
